(19) United States
(12) Patent Application Publication
(10) Pub. No.: US 2014/0170669 A1
(43) Pub. Date: Jun. 19, 2014

(54) DEVICES FOR TARGET DETECTION AND METHODS OF USE THEREOF

(71) Applicant: NanoMR, Inc., Albuquerque, MN (US)

(72) Inventor: Jaclyn D. Vandervest, Albuquerque, NM (US)

(73) Assignee: NanoMR, Inc., Albuquerque, NM (US)

(21) Appl. No.: 14/132,770

(22) Filed: Dec. 18, 2013

Related U.S. Application Data
(60) Provisional application No. 61/739,575, filed on Dec. 19, 2012.

(51) Int. Cl. G01N 33/569

(52) U.S. Cl. CPC G01N 33/56938; G01N 33/56911
USPC 435/6.15; 435/287.2; 435/7.32; 435/7.33

(57) ABSTRACT

The invention generally relates to devices for target detection and methods of use thereof.

### RELATED APPLICATIONS

The present application claims the benefit of and priority to U.S. provisional patent application Ser. No. 61/739,575 filed Dec. 19, 2012, the content of which is incorporated by reference herein in its entirety.

### FIELD OF THE INVENTION

The invention generally relates to devices for target detection and methods of use thereof.

### BACKGROUND

Contamination is a problem in food and drug industries as well as in environmental health and national security. The potential for a contaminant to go undetected in food, drugs, or the environment poses significant threats to human safety. To address those threats, organizations such as the United States Pharmacopoeia (USP) have issued guidelines setting acceptable limits of microbial contamination. Testing those microbial limits, sometimes called bioburden testing, is an essential part of making and selling pharmaceuticals, medical devices, and food.

Traditional testing methods are problematic because they are slow and have limited sensitivity. For example, many tests are unable to detect very low numbers of molecules or organisms and thus require a target to be concentrated or grown in a culture. Culturing microbes on agar plates can require days or even weeks. Molecular tests based on technologies such as polymerase chain reaction or enzyme-linked immunosorbent assays generally require an enrichment step that can require 24 to 48 hours or longer.

Those tests impose large costs on industry as products must stand by unsold and unused while the tests are performed. If a slow-growing bacterium requires a 76-hour enrichment culture, for example, many truckloads' worth of product may have to sit idly in warehouses for those days. Nevertheless, the safety of our food and drug supply requires that accurate and reliable tests be performed.

### SUMMARY

The invention provides rapid, sensitive, and accurate tests for chemical and microbial contamination of products and facilities. Methods and device of the invention are capable of detecting very small amounts of contamination, as low as a single target in a milliliter of sample. Sensitive detection is accomplished by introducing a molecular binding moiety to the sample and allowing it to bind to the target and then separating the binder/target complex from the remainder of the sample. Speed and sensitivity is maximized by the techniques, devices, and reagents employed.

A binding buffer may be used to flush the sample into the separation chamber and is chemically optimized to encourage binding of target while preventing non-specific aggregation of particles in the sample. The binding buffer may be replaced with a wash buffer to further remove non-target materials from the bound target. The entire contaminant capture procedure can be performed rapidly within a fluidic device, allowing the target contaminant to be detected from a very low starting concentration within a short period of time. Since low concentrations of contaminant can be detected very rapidly, methods and device of the invention can be used for bioburden or microbial limit testing in settings throughout the food and drug industries. Since testing is rapid and accurate, food and drugs may be produced and distributed safely, while avoiding high costs associated with prior art tests.

In some aspects, the invention provides a method for detecting a target analyte in a sample. The method includes transferring the sample from a collection tube into a chamber on a device, flushing the collection tube with a liquid that includes a binding moiety, and delivering the liquid comprising the binding moiety into the chamber. The target analyte is isolated from the sample and analyzed.

Isolating the target analyte can employ a binding moiety that is linked to a magnetic particle, such as superparamagnetic particle with a diameter less than about 250 nm. Magnetic separation techniques can be used to wash away the sample. For example, where the binding moiety is an antibody, it may bind specifically to the target analyte, allowing other components of the sample to be washed away. The sample may be analyzed by optical detection, non-optical detection, or other methods. For example, nucleic acid may be extracted from the sample and analyzed by PCR, sequencing, hybridization to probes, or other methods. In some embodiments, the DNA extraction occurs within the device.

The device can include a fluidic chip or cartridge. A pneumatic interface may be included and materials can be transferred through, stored within, or flushed from different channels and chambers of the device by pneumatic pressure. The liquid that includes the binding moiety may include a binding buffer—a buffer that is formulated to enhance binding of the binding moiety to a target analyte and reduce formation of particle aggregates, e.g., a solution comprising a moiety that specifically binds the target. After the target analyte is captured by the binding moiety, it may optionally be washed with a wash solution, for example, to remove non-specifically bound components from the binding moiety.

Methods of the invention can be rapid, with all steps taking less than 24 hours. In some embodiments, the steps take less than about 12 hours, 6 hours, or 3 hours.

In related aspects, the invention provides a method for detecting a target analyte by performing the following steps within a fluidic device. A binding buffer, a binding moiety, and a sample containing a target analyte are delivered into a chamber on the device. The sample is incubated until the binding moiety binds to the target analyte. The target analyte is isolated and analyzed. The method can include loading a collection tube holding the sample into or onto the device and using pressure to deliver the sample into the chamber. The binding moiety, the buffer, or both may optionally be flushed through the collection tube, for example, while on the device, and into the chamber to ensure very sensitive detection.

The target analyte, bound to the binding moiety, is retained within a portion of the device. There, it can be washed with the binding buffer, water, a wash solution, or a combination thereof. That portion of the device may be the chamber in which the sample was initially incubated, may be a second chamber, or both. In some embodiments, all of the steps of the method can be performed in less than 12 hours, for example, in less than 4 hours or 2 hours.

Isolation of the target analyte may employ properties of the binding moiety. For example, the binding moiety may be bound to a solid substrate such as a wall of a fluidic channel or chamber, a surface of a material in a column, or particles such as beads. The particles can be magnetic particles such as small magnetic particles that include a paramagnetic material, have a diameter of less than about 250 nm, or both. The binding moiety can be an antibody, a ligand, streptavidin, biotin, or any other suitable binding molecule. In certain embodiments, the target analyte is a microorganism, such as a pathogenic bacterium. An antibody may be used to bind to an antigen on a cell surface of the organism.

In other aspects, the invention provides methods for detecting a target analyte that involve introducing a sample containing a target analyte and a plurality of beads each linked to a binding moiety into a chamber on a device and isolating the target analyte from the sample within the chamber. The target analyte is further transferred into a concentrator chamber on the device, concentrated there, and analyzed. The beads may be small magnetic beads (e.g., d<300 nm). Pressure is used to introduce the sample into the chamber and transfer the target analyte into the concentrator chamber. Pneumatic pressure may be used.

In some embodiments, the beads are suspended in a liquid that includes a binding buffer formulated to enhance binding of the binding moiety to the target analyte, to reduce formation of particle aggregates, or both. Further, non-specifically bound components may be washed from the target, for example, using the binding buffer, using a wash buffer, using water, or a combination thereof. All of the steps may be performed in less than a day, e.g., half a day or a few hours.

In other related aspects, the invention provides a device for detecting a target analyte. The device includes an ampoule containing a plurality of beads linked to binding moieties, a reservoir of binding buffer, and a network of fluidic channels configured to transfer a sample, the beads, and the binding buffer into a chamber on the device. The device further includes a mechanism to retain the bead-bound target analyte in the chamber while buffer is removed and replaced by a wash buffer. The device also provides a vessel for collection of the target analyte. In some embodiments, the device is provided with a mechanism to rupture the ampoule. Fluidic channels may be used to conduct sample or reagents within the device. For example, in some embodiments, macrofluidic channels conduct the sample into the chamber, microfluidic channels transfer the target analyte into the collection vessel, or both. The device may also include a magnetic concentrator configured for fluid communication with the chamber.

### BRIEF DESCRIPTION OF THE DRAWINGS

FIG. 1 illustrates detecting an analyte according to certain embodiments.

FIG. 2 shows embodiments of methods of detecting a target analyte.

FIG. 3 shows a device according to embodiments of the invention.

FIG. 4 gives a perspective view of the device shown in FIG. 3.

FIG. 5 diagrams components of a device of the invention.

### DETAILED DESCRIPTION

The present invention provides methods and devices for microbial limit and bioburden testing in an industrial, environmental, or biological sample. The invention allows the rapid detection of chemical or microbial contaminants at very low levels in the sample, thus allowing early and accurate detection and identification of the contaminants. Since methods of the invention are able to isolate target analytes at very low levels, methods of the invention reduce or eliminate culturing or enrichment steps that are typically associated with bioburden testing and allow for more rapid analysis of the target. In this manner, methods of the invention provide data that organizations may use to assure the fitness of their products for commercial distribution.

Moreover, methods and devices of the invention may be used for environmental quality sampling (e.g., to make pollution remediation recommendations) or national security purposes (e.g., to detect anthrax).

The invention generally relates to conducting an assay on a sample that isolates a target analyte from the sample and allows for analysis of the analyte with minimal (e.g., less than 24 hours of) or no culturing of the pathogen. In certain embodiments, methods of the invention involve obtaining a sample including a target analyte, conducting an assay that isolates the target analyte from the sample, and analyzing the pathogen. In some embodiments, methods include a short culturing or enrichment step, for example, a step that is shorter than a few hours. In particular embodiments, there is no culturing or enriching step and the isolated target analyte is analyzed directly without any culturing enrichment.

Any sample may be collected and assayed using methods and device of the invention. For example, samples can be taken of pharmaceutical or chemical compounds. Samples can be taken from the ambient air or surfaces, for example, of structures, equipment, devices such as medical devices, animals, or people. Biological samples can be taken such as a bodily tissue or fluid.

In some embodiments, the sample is an air sample, for example, as collected with a volumetric sampler that is capable of collecting a sufficient volume of air (e.g., about 100 liters to about 1,000 liters). A volumetric air sampler may include a blower and a filter. Air is drawn into the sampler and through a glass fiber or quartz filter, so that matter collects on the filter surface. The filter can then be immersed in liquid, such as a buffer, a nutrient medium (e.g., trypticase soy) or an alcohol, within, for example, a collection tube. Air sampling may collect particulate matter, microbes, insects, aerosols such as oil mists or fog droplets, exhaled breath, or other constituents of the atmosphere. Suitable air samplers are discussed in U.S. Pat. No. 8,171,803; U.S. Pat. No. 7,046,011; U.S. Pat. No. 6,087,183; U.S. Pat. No. 5,467,776; and U.S. Pat. No. 5,201,231, incorporated by reference.

A sample may be collected from a surface. Surface sampling techniques for bioburden or microbial limit testing are known in the art and any suitable method can be used. For example, in some embodiments, the replicate organism detection and counting (RODAC) method—also known as the contact plate method—is used for surface sampling. The contact plate method involves pressing an over-filled Petri dish against the surface to be tested. The dish can then be cultured or scraped directly into a collection tube. The contact plate method is described in Hall and Hartnett, 1964, Measurement of bacterial contamination on surfaces, Pub Health Report 79:1012-1024; U.S. Pat. No. 3,787,290; U.S. Pat. No. 3,337,416. In certain embodiments, a sterile swab is unwrapped and a surface is swabbed. The swab is then enclosed in a collection tube, optionally immersed therein in a liquid such as a buffer, a nutrient medium, or an alcohol. Surface sampling techniques are described in U.S. Pat. No. 7,611,862; U.S. Pat. No. 5,859,375; U.S. Pat. No. 5,823,592; and U.S. Pub. 2010/0313685, the contents of each of which are incorporated by reference.

In some embodiments, the sample is a bodily fluid. A bodily fluid refers to a liquid material derived from, for example, a human or other mammal. Such bodily fluids include, without limit, mucus, blood, plasma, serum, serum derivatives, bile, phlegm, saliva, sweat, amniotic fluid, mammary fluid, urine, sputum, and cerebrospinal fluid (CSF), such as lumbar or ventricular CSF. A bodily fluid may also be a fine needle aspirate. A bodily fluid may also be media containing cells or biological material.

Methods of the invention involve collecting a sample having a target analyte in a container, such as a sample collection tube like those sold under the trademark VACUTAINER (collection vial, commercially available by BD, Franklin Lakes, N.J.). In certain embodiments, a solution is added that prevents or reduces aggregation of endogenous aggregating factors, such as heparin in the case of blood. A particular advantage of the methods described herein is the ability to capture and isolate different unknown pathogens directly from samples at low concentrations that are characteristic of clinical samples (as low as 1 CFU/ml of bacteria in a blood sample).

Methods of the invention may be used to detect any target analyte. The target analyte refers to the substance in the sample that will be captured and isolated by methods of the invention. The target analyte may be inorganic (e.g., a metal, a cyanide or cyanate, a salt, etc.) or organic chemicals, macromolecules (chitin, peptidoglycan, carbohydrates, proteins, nucleic acids, lipids, etc.,), bacteria, fungi, a cell (such as a cancer cell, a white blood cell a virally infected cell, or a fetal cell circulating in maternal circulation), a virus, a nucleic acid (e.g., DNA or RNA), a receptor, a ligand, a hormone, a drug, a chemical substance, or any molecule known in the art. In certain embodiments, the target is a pathogenic bacteria. In other embodiments, the target is a gram positive or gram negative bacteria. Exemplary bacteria that may be captured and isolated by methods of the invention include those of the genera Alphaproteobacteria, Bacillus, Betaproteobacteria, Bifidobacterium, Borrelia, Campylobacter, Candida, Citrobacter, Clostridium, Enterobacter, Enterococcus, Escherichia, Flavobacterium, Fusobacterium, Gammaproteobacteria, Klebsiella, Kluyvera, Lactobacillus, Legionella, Leuconostoc, Listeria, Micrococcus, Mycobacterium, Neisseriaceae, Pediococcus, Pneumococcus, Porphyromonas, Prevotella, Propionibacterium, Proteus, Rhodospirillum, Rickettsia, Saccharomyces, Salmonella, Serratia, Shigella, Sphaerotilus, Staphylococcus, Streptococcus, Thermoanaerobacter, Thermoproteus, Vibrio, and Yersinia, or a combination thereof.

A particular advantage of methods of the invention is for capture and isolation of bacteria and fungi directly from blood samples at low concentrations that are present in many clinical samples (as low as 1 CFU/ml of bacteria in a blood sample).

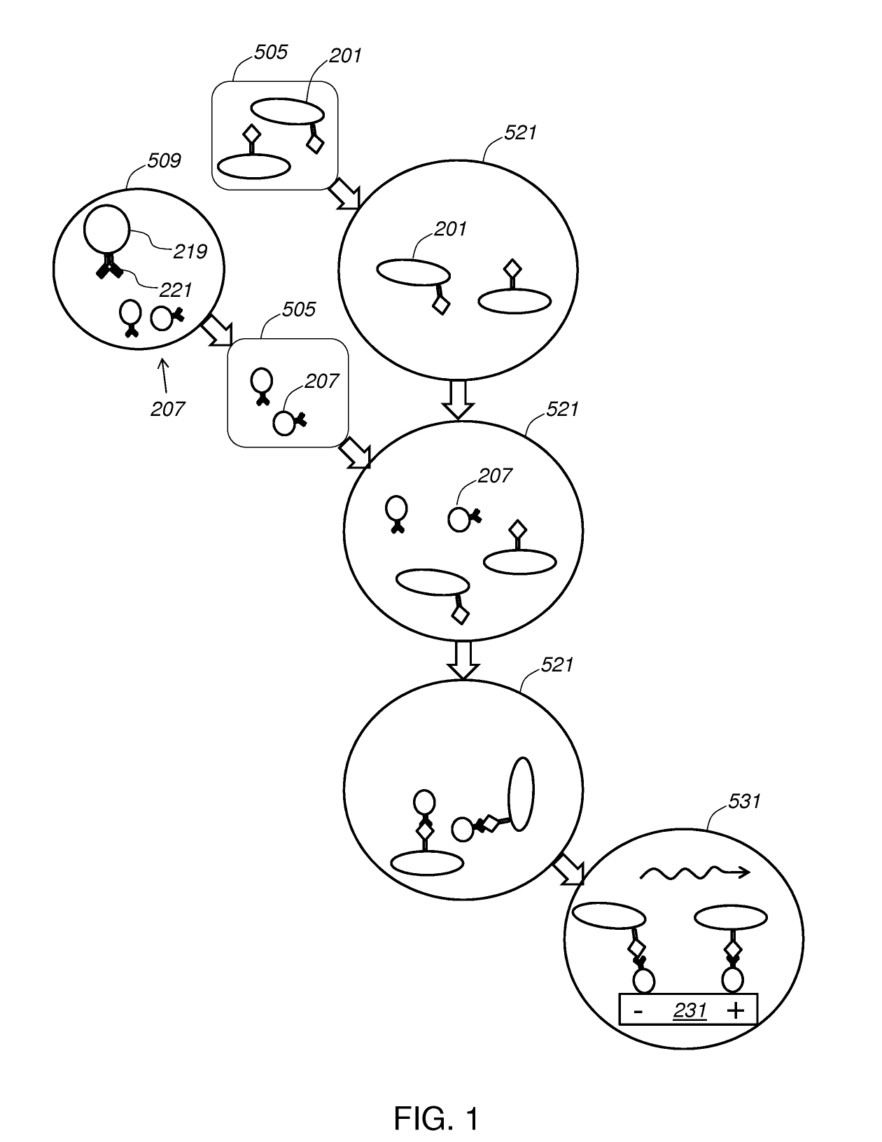

FIG. 1 shows a method for detecting a target analyte 201 in a sample. The sample is transferred from a collection tube 505 into a chamber 521 (e.g., on a fluidic device). Chamber 505 is flushed with a liquid containing a binding buffer 207 (e.g., a solution comprising a moiety that binds to a target), and that liquid is delivered into chamber 521. The binding buffer 207 includes particles 219 used to isolate target analyte 207 from the sample. Isolation can take place within chamber 521, or the bound target analyte 201 may be transferred to another chamber 531 on the device.

In some embodiments, the particles 219 in binding buffer 207 are magnetic particles. The sample is mixed with magnetic particles 219 having a particular magnetic moment and also including a target-specific binding moiety 221 to generate a mixture that is allowed to incubate such that the particles bind to a target analyte 201 in the sample, such as a bacterium in a blood sample. The mixture is allowed to incubate for a sufficient time to allow for the particles to bind to the target analyte. The process of binding magnetic particles 219 to the target analyte 201 associates a magnetic moment with the target analytes 201, and thus allows the target analytes to be manipulated through forces generated by magnetic fields upon the attached magnetic moment.

As will be shown below, transfer of the sample into a chamber 521 and flushing collection tube 505 with binding buffer 207 may take place on a fluidic device or system. Chamber 521 and collection tube 505 may be in fluid communication by one or more channels. Liquids may be transferred among chambers and channels through the use of pressure, such as pressure applied through the use of another liquid (e.g., an immiscible liquid) or pneumatic pressure.

In general, incubation time (e.g., in chamber 521) will depend on the desired degree of binding between the target analyte and the magnetic beads (e.g., the amount of moment that would be desirably attached to the target), the amount of moment per target, the amount of time of mixing, the type of mixing, the reagents present to promote the binding and the binding chemistry system that is being employed. Incubation time can be anywhere from about 5 seconds to a few days. Exemplary incubation times range from a few seconds to about 2 hours. Incubation times can be optimized to allow the entire isolation method to take place within a specific time, e.g., 6 hours, 3 hours, 1 hour, 30 minutes, or 10 minutes, or a few minutes. Binding occurs over a wide range of temperatures, generally between 15° C. and 40° C.

In certain aspects, methods of the invention involve introducing magnetic particles 219 including a target-specific binding moiety 221 to a sample in order to create a mixture, incubating the mixture to allow the particles 207 to bind to a target 201, applying a magnetic field to capture target/magnetic particle complexes on a surface, thereby isolating target/magnetic particle complexes. Methods of the invention may further involve incubating the mixture in a binding solution that facilitates binding, washing the mixture in a wash solution that reduces particle aggregation, using a solution that facilitates binding and reduce aggregation, or a combination thereof. Certain fundamental technologies and principles are associated with binding magnetic materials to target entities and subsequently separating by use of magnet fields and gradients. Such fundamental technologies and principles are known in the art and have been previously described, such as those described in Murphy, 2011, Janeway's Immunobiology 8 Ed, Garland Science (New York, N.Y.), 888 pages, the contents of which are incorporated by reference herein. Methods of producing suitable magnetic particles are known in the art. See for example U.S. Pat. No. 5,597,531; U.S. Pat. No. 4,230,685; U.S. Pat. No. 4,677,055; U.S. Pat. No. 4,695,393; U.S. Pat. 5,695,946; U.S. Pat. No. 4,018,886; U.S. Pat. No. 4,267,234; U.S. Pat. No. 4,452,773; U.S. Pat. No. 4,554,088; U.S. Pat. No. 4,659,678; U.S. Pat. No. 5,186,827; U.S. Pat. No. 4,795,698, the contents of each of which are incorporated by reference.

Methods of the invention may use any magnetic particles 219. In certain embodiments, methods of the invention are performed with magnetic particle having a magnetic moment that allows for isolation of as low as 1 CFU/ml of bacteria in the sample. Production of magnetic particles 219 is further shown for example in U.S. Pat. No. 3,970,518 and in Int. Pat. Pub. WO 91/02811, the content of each of which is incorporated by reference herein in its entirety.

Any type of magnetic particle may be used for substrate 219 in accordance with the invention. Methods of the invention include use of diamagnetic materials, paramagnetic materials, superparamagnetic materials, ferromagnetic materials, or a combination thereof (independently or in combination). Diamagnetic materials are slightly repelled by a magnetic field and the material does not retain the magnetic properties when the external field is removed. Paramagnetic materials (e.g., aluminum or platinum) are slightly attracted by a magnetic field and the material does not retain the magnetic properties when the external field is removed. Superparamagnetic materials are much more susceptible to magnetization than paramagnetic materials. See Gittleman et al., 1974, Superparamagnetism and relaxation effects in granular Ni—SiO₂ and Ni—Al₂O₃ films, Phys Rev B 9:3891-3897. Ferromagnetic materials (e.g., iron or nickel) exhibit a strong attraction to magnetic fields and are able to retain their magnetic properties after the external field has been removed.

Further, magnetic properties of substrate 219 may depend on the size of the particles of substrate. Some ferromagnetic materials, e.g., magnetic iron oxide, may be characterized as superparamagnetic when provided in crystals of about 30 nm or less in diameter. Larger crystals of ferromagnetic materials, by contrast, retain permanent magnet characteristics after exposure to a magnetic field and tend to aggregate thereafter due to strong particle-particle interaction. In certain embodiments, the particles are superparamagnetic particles. In certain embodiments, the magnetic particle is an iron containing magnetic particle. In other embodiments, the magnetic particle includes iron oxide or iron platinum.

In certain embodiments, the magnetic particles include at least about 10% superparamagnetic beads by weight, at least about 20% superparamagnetic beads by weight, at least about 30% superparamagnetic beads by weight, at least about 40% superparamagnetic beads by weight, at least about 50% superparamagnetic beads by weight, at least about 60% superparamagnetic beads by weight, at least about 70% superparamagnetic beads by weight, at least about 80% superparamagnetic beads by weight, at least about 90% superparamagnetic beads by weight, at least about 95% superparamagnetic beads by weight, or at least about 99% superparamagnetic beads by weight. In a particular embodiment, the magnetic particles include at least about 70% superparamagnetic beads by weight.

In certain embodiments, the superparamagnetic beads are less than 100 nm in diameter. In other embodiments, the superparamagnetic beads are about 150 nm in diameter, are about 200 nm in diameter, are about 250 nm in diameter, are about 300 nm in diameter, are about 350 nm in diameter, are about 400 nm in diameter, are about 500 nm in diameter, or are about 1000 nm in diameter. In a particular embodiment, the superparamagnetic beads are from about 100 nm to about 250 nm in diameter.

In certain embodiments, the particles 219 are beads (e.g., nanoparticles) that incorporate magnetic materials, or magnetic materials that have been functionalized, or other configurations as are known in the art. In certain embodiments, nanoparticles may be used that include a polymer material that incorporates magnetic material, such as nanometal material. When those nanometal material or crystals, such as Fe₃O₄, are superparamagnetic, they may provide advantageous properties, such as being capable of being magnetized by an external magnetic field, and demagnetized when the external magnetic field has been removed. This may be advantageous for facilitating sample transport into and away from an area where the sample is being processed without undue bead aggregation.

One or more or many different nanometal may be employed, such as Fe₃O₄, FePt, or Fe, in a core-shell configuration to provide stability, and/or various others as may be known in the art. In many applications, it may be advantageous to have a nanometal having as high a saturated moment per volume as possible, as this may maximize gradient related forces, and/or may enhance a signal associated with the presence of the beads. It may also be advantageous to have the volumetric loading in a bead be as high as possible, for the same or similar reasons. In order to maximize the moment provided by a magnetizable nanometal, a certain saturation field may be provided. For example, for Fe₃O₄ superparamagnetic particles 219, this field may be on the order of about 0.3 T.

The size of the nanometal-containing bead 219 may be optimized for a particular application, for example, maximizing moment loaded upon a target, maximizing the number of beads 219 on a target with an acceptable detectability, maximizing desired force-induced motion, and/or maximizing the difference in attached moment between the labeled target and non-specifically bound targets or bead aggregates or individual beads. While maximizing is referenced by example above, other optimizations or alterations are contemplated, such as minimizing or otherwise desirably affecting conditions.

In an exemplary embodiment, a polymer bead containing 80 wt % Fe₃O₄ super-paramagnetic particles, or for example, 90 wt % or higher super-paramagnetic particles, is produced by encapsulating superparamagnetic particles with a polymer coating to produce a bead 219 having a diameter of about 250 nm.

With continued reference to FIG. 1, magnetic particles 219 for use with methods of the invention have a target-specific binding moiety 221 that allows for the particles 219 to specifically bind the target analyte 201 of interest in the sample. The target-specific moiety 221 may be any molecule known in the art and will depend on the target 201 to be captured and isolated. Exemplary target-specific binding moieties include nucleic acids, proteins, ligands, antibodies, aptamers, and receptors. In some embodiments, target-specific moiety 221 includes a chelator.

In particular embodiments, the target-specific binding moiety 221 is an antibody, such as an antibody that binds a particular bacterium. General methodologies for antibody production, including criteria to be considered when choosing an animal for the production of antisera, are described in Kontermann, 2010, Antibody Engineering Volume 1 2Ed, Springer-Verlag (Berlin Heidelberg) 800 pages, and Harlow, 1988, Antibodies: A Laboratory Manual, Cold Spring Harbor Laboratory Press (Cold Spring Harbor, N.Y.) 726 pages. For example, an animal of suitable size such as goats, dogs, sheep, mice, or camels are immunized by administration of an amount of immunogen, such the target bacteria, effective to produce an immune response. An exemplary protocol is as follows. The animal is injected with 100 milligrams of antigen resuspended in adjuvant, for example Freund's complete adjuvant, dependent on the size of the animal, followed three weeks later with a subcutaneous injection of 100 micrograms to 100 milligrams of immunogen with adjuvant dependent on the size of the animal, for example Freund's incomplete adjuvant. Additional subcutaneous or intraperitoneal injections every two weeks with adjuvant, for example Freund's incomplete adjuvant, are administered until a suitable titer of antibody in the animal's blood is achieved. Exemplary titers include a titer of at least about 1:5000 or a titer of 1:100,000 or more, i.e., the dilution having a detectable activity. The antibodies are purified, for example, by affinity purification on columns containing protein G resin or target-specific affinity resin.

The technique of in vitro immunization of human lymphocytes is used to generate monoclonal antibodies. Such techniques are known in the art. See, e.g., Mulder et al., 1993, Characterization of two human monoclonal antibodies reactive with HLA-B12 and HLA-B60, respectively, raised by in vitro secondary immunization of peripheral blood lymphocytes, Hum. Immunol 36(3):186-192; Stauber et al., 1993, Rapid generation of monoclonal antibody-secreting hybridomas against African horse sickness virus by in vitro immunization and the fusion/cloning technique, J Immunol Methods 161(2):157-168; and Venkateswaran, et al., 1992, Production of anti-fibroblast growth factor receptor monoclonal antibodies by in vitro immunization, Hybridoma, 11(6):729-739. These techniques can be used to produce antigen-reactive monoclonal antibodies, including antigen-specific IgG and IgM monoclonal antibodies.

Any antibody or fragment thereof having affinity and specific for the target of interest is within the scope of the invention provided herein. Immunomagnetic beads against Salmonella are provided in Vermunt et al. (J. Appl. Bact. 72:112, 1992). Immunomagnetic beads against Staphylococcus aureus are provided in Johne et al. (J. Clin. Microbiol. 27:1631, 1989). Immunomagnetic beads against Listeria are provided in Skjerve et al. (Appl. Env. Microbiol. 56:3478, 1990). Immunomagnetic beads against Escherichia coli are provided in Lund et al. (J. Clin. Microbiol. 29:2259, 1991).

Methods for attaching the target-specific binding moiety 221 to the magnetic particle 219, including the coating of particles with antibodies, are known in the art. See for example Kontermann, 2010, Antibody Engineering Volume 1 2Ed, Springer-Verlag (Berlin Heidelberg) 800 pages; Harlow, 1988, Antibodies: A Laboratory Manual, Cold Spring Harbor Laboratory Press (Cold Spring Harbor, N.Y.) 726 pages; and Stanley, 2002, Essentials in Immunology and Serology, Delmar Cengage (Independence, Ky.) 560 pages; U.S. Pat. No. 7,699,979, the contents of each of which are incorporated by reference. Such methodology can easily be modified by one of skill in the art to bind other types of target-specific binding moieties 221 to magnetic particles 219. In addition, certain types of magnetic particles coated with a functional moiety are commercially available from Sigma-Aldrich (St. Louis, Mo.).

In certain embodiments, a buffer solution 207 is added to the sample along with the magnetic beads 219. An exemplary buffer includes Tris(hydroxymethyl)-aminomethane hydrochloride at a concentration of about 75 mM. It has been found that the buffer composition, mixing parameters (speed, type of mixing, such as rotation, shaking etc., and temperature) influence binding. It is important to maintain osmolality of the final solution (e.g., blood+buffer) to maintain high label efficiency. In certain embodiments, buffers used in methods of the invention are designed to prevent lysis of blood cells, facilitate efficient binding of targets with magnetic beads and to reduce formation of bead aggregates. It has been found that the buffer solution containing 300 mM NaCl, 75 mM Tris-HCl pH 8.0 and 0.1% Tween 20 meets these design goals.

Without being limited by any particular theory or mechanism of action, it is believed that sodium chloride is mainly responsible for maintaining osmolality of the solution and for the reduction of non-specific binding of magnetic bead through ionic interaction. Tris(hydroxymethyl)-aminomethane hydrochloride is a well-established buffer compound frequently used in biology to maintain pH of a solution. It has been found that 75 mM concentration is beneficial and sufficient for high binding efficiency. Likewise, Tween 20 is widely used as a mild detergent to decrease nonspecific attachment due to hydrophobic interactions. Various assays use Tween 20 at concentrations ranging from 0.01% to 1%. The 0.1% concentration appears to be optimal for the efficient labeling of bacteria, while maintaining blood cells intact.

An alternative approach to achieve high binding efficiency while reducing time required for the binding step is to use static mixer, or other mixing devices that provide efficient mixing of viscous samples at high flow rates, such as at or around 5 mL/min. In one embodiment, the sample is mixed with binding buffer in ratio of, or about, 1:1, using a mixing interface connector. The diluted sample then flows through a mixing interface connector where it is mixed with target-specific nanoparticles. Additional mixing interface connectors providing mixing of sample and antigen-specific nanoparticles can be attached downstream to improve binding efficiency. The combined flow rate of the labeled sample is selected such that it is compatible with downstream processing.

After binding of the magnetic particles 219 to the target analyte 201 in the mixture to form target/magnetic particle complexes, a magnetic field is applied to the mixture to capture the complexes on a surface (e.g., using magnet 231 as shown in FIG. 1). Components of the mixture that are not bound to magnetic particles will not be affected by the magnetic field and will remain free in the mixture. Methods and apparatuses for separating target/magnetic particle complexes from other components of a mixture are known in the art. For example, a steel mesh may be coupled to a magnet, a linear channel or channels may be configured with adjacent magnets, or quadrapole magnets with annular flow may be used. Other methods and apparatuses for separating target/magnetic particle complexes from other components of a mixture are shown in Rao et al. (U.S. Pat. No. 6,551,843), Liberti et al. (U.S. Pat. No. 5,622,831), Hatch et al. (U.S. Pat. No. 6,514,415), Benjamin et al. (U.S. Pat. No. 5,695,946), Liberti et al. (U.S. Pat. No. 5,186,827), Wang et al. (U.S. Pat. No. 5,541,072), Liberti et al. (U.S. Pat. No. 5,466,574), and Terstappen et al. (U.S. Pat. No. 6,623,983), the content of each of which is incorporated by reference herein in its entirety.

With continued reference to FIG. 1, the magnetic capture is achieved at high efficiency by utilizing a flow-through capture cell 531 with a number of strong rare earth bar magnets 231 placed perpendicular to the flow of the sample. When using a flow chamber with flow path cross-section 0.5 mm×20 mm (h×w) and 7 bar NdFeB magnets, the flow rate could be as high as 5 mL/min or more, while achieving capture efficiency close to 100%.

The above described type of magnetic separation produces efficient capture of a target analyte and the removal of a majority of the remaining components of a sample mixture. However, such a process may produce a sample that contains a percent of magnetic particles 219 that are not bound to target analytes 201, as well as non-specific target entities. Non-specific target entities may for example be bound at a much lower efficiency, for example 1% of the surface area, while a target of interest might be loaded at 50% or nearly 100% of the available surface area or available antigenic cites. However, even 1% loading may be sufficient to impart force necessary for trapping in a magnetic gradient flow cell or sample chamber.

The presence of magnetic particles 219 that are not bound to target analytes 201 and non-specific target entities on the surface that includes the target/magnetic particle complexes may interfere with the ability to successfully detect the target of interest. The magnetic capture of the resulting mix, and close contact of magnetic particles 219 with each other and bound targets 201, result in the formation of aggregate that is hard to dispense and which might be resistant or inadequate for subsequent processing or analysis steps. In order to remove magnetic particles that are not bound to target analytes and non-specific target entities, methods of the invention may further involve washing the surface with a wash solution that reduces particle aggregation, thereby isolating target/magnetic particle complexes from the magnetic particles that are not bound to target analytes and non-specific target entities. The wash solution minimizes the formation of the aggregates.

Methods of the invention may use any wash solution that imparts a net negative charge to the magnetic particle that is not sufficient to disrupt interaction between the target-specific moiety 221 of the magnetic particle 219 and the target analyte 201. Without being limited by any particular theory or mechanism of action, it is believed that attachment of the negatively charged molecules in the wash solution to magnetic particles provides net negative charge to the particles and facilitates dispersal of non-specifically aggregated particles. At the same time, the net negative charge is not sufficient to disrupt strong interaction between the target-specific moiety of the magnetic particle and the target analyte (e.g., an antibody-antigen interaction). Exemplary solutions include heparin, Tris-HCl, Tris-borate-EDTA (TBE), Tris-acetate-EDTA (TAE), Tris-cacodylate, HEPES (4-(2-hydroxyethyl)-1-piperazineethanesulphonic acid), PBS (phosphate buffered saline), PIPES (piperazine-N,N′-bis(2-ethanesulfonic acid), MES (2-N-morpholino)ethanesulfonic acid), Tricine (N-(Tri(hydroxymethyl)methyl)glycine), and similar buffering agents. In certain embodiments, only a single wash cycle is performed. In other embodiments, more than one wash cycle is performed.

In particular embodiments, the wash solution includes heparin. For embodiments in which the body fluid sample is blood, the heparin also reduces probability of clotting of blood components after magnetic capture. The bound targets 201 are washed with heparin-containing buffer 1-3 times to remove blood components and to reduce formation of aggregates.

Since methods of the invention are able to isolate pathogens at very low levels, methods of the invention reduce or eliminate the culturing step that is typically associated with pathogen analysis and allow for more rapid analysis of the isolated pathogen. Reduced culturing refers to culturing for at most about 24 hours, for example, for at most about 23 hours, for at most about 22 hours for at most about 21 hours, for at most about 20 hours, for at most about 15 hours, for at most about 10 hours, for at most about 9 hours, for at most about 8 hours, for at most about 7 hours, for at most about 6 hours, for at most about 5 hours, for at most about 4 hours, for at most about 3 hours, for at most about 2 hours, or for at most about 1 hr. In particular embodiments, culturing is for less than 1 hr., for example, about 45 minutes, about 30 minutes, about 15 minutes, about 10 minutes, or for less than 10 minutes. In particular embodiments, culturing is completely eliminated and the isolated bacteria is analyzed directly without any culturing, i.e., culturing is eliminated.

The target may be analyzed by a multitude of existing technologies, such as nuclear magnetic resonance (NMR), miniature NMR, Polymerase Chain Reaction (PCR), mass spectrometry, fluorescent labeling and visualization using microscopic observation, fluorescent in situ hybridization (FISH), growth-based antibiotic sensitivity tests, and variety of other methods that may be conducted with purified target without significant contamination from other sample components. Analysis using NMR is described in U.S. Pub. 2011/0262925, herein incorporated by reference in its entirety. In one embodiment, isolated cells are lysed with a chaotropic solution, and DNA is bound to DNA extraction resin. After washing of the resin, the DNA may be eluted and used in quantitative RT-PCR to detect the presence or an identity of the cells, such as detecting genera, species, or subclasses of bacteria.

In another embodiment, captured bacteria is removed from the magnetic particles 219 to which they are bound and the processed sample is mixed with a detectable label such as fluorescent labeled antibodies specific to the bacteria or fluorescent Gram stain. After incubation, the reaction mixture is filtered through 0.2 μm to 1.0 μm filter to capture labeled bacteria while allowing majority of free beads and fluorescent labels to pass through the filter. Bacteria is visualized on the filter using microscopic techniques, e.g. direct microscopic observation, laser scanning or other automated methods of image capture. The presence of bacteria is detected through image analysis. After the positive detection by visual techniques, the bacteria can be further characterized using PCR or genomic methods.

Detection of bacteria of interest can be performed by use of nucleic acid probes following procedures which are known in the art. Suitable procedures for detection of bacteria using nucleic acid probes are described, for example, in Stackebrandt et al. (U.S. Pat. No. 5,089,386), King et al. (WO 90/08841), Foster et al. (WO 92/15883), and Cossart et al. (WO 89/06699), each of which is hereby incorporated by reference.

A suitable nucleic acid probe assay generally includes sample treatment and lysis, hybridization with selected probe(s), hybrid capture, and detection. Lysis of the bacteria is necessary to release the nucleic acid for the probes. The nucleic acid target molecules are released by treatment with any of a number of lysis agents, including alkali (such as NaOH), guanidine salts (such as guanidine thiocyanate), enzymes (such as lysozyme, mutanolysin and proteinase K), and detergents. Lysis of the bacteria, therefore, releases both DNA and RNA, particularly ribosomal RNA and chromosomal DNA both of which can be utilized as the target molecules with appropriate selection of a suitable probe. Use of rRNA as the target molecule(s), may be advantageous because rRNAs constitute a significant component of cellular mass, thereby providing an abundance of target molecules. The use of rRNA probes also enhances specificity for the bacteria of interest, that is, positive detection without undesirable cross-reactivity which can lead to false positives or false detection.

Hybridization includes addition of the specific nucleic acid probes. In general, hybridization is the procedure by which two partially or completely complementary nucleic acids are combined, under defined reaction conditions, in an anti-parallel fashion to form specific and stable hydrogen bonds. The selection or stringency of the hybridization/reaction conditions is defined by the length and base composition of the probe/target duplex, as well as by the level and geometry of mis-pairing between the two nucleic acid strands. Stringency is also governed by such reaction parameters as temperature, types and concentrations of denaturing agents present and the type and concentration of ionic species present in the hybridization solution.

The hybridization phase of the nucleic acid probe assay is performed with a single selected probe or with a combination of two, three or more probes. Probes are selected having sequences which are homologous to unique nucleic acid sequences of the target organism. In general, a first capture probe is utilized to capture formed hybrid molecules. The hybrid molecule is then detected by use of antibody reaction or by use of a second detector probe which may be labeled with a radioisotope (such as phosphorus-32) or a fluorescent label (such as fluorescein) or chemiluminescent label.

Detection of bacteria of interest can also be performed by use of PCR techniques. A suitable PCR technique is described, for example, in Verhoef et al. (WO 92/08805). Such protocols may be applied directly to the bacteria captured on the magnetic beads. The bacteria is combined with a lysis buffer and collected nucleic acid target molecules are then utilized as the template for the PCR reaction.

For detection of the selected bacteria by use of antibodies, isolated bacteria are contacted with antibodies specific to the bacteria of interest. As noted above, either polyclonal or monoclonal antibodies can be utilized, but in either case have affinity for the particular bacteria to be detected. These antibodies, will adhere/bind to material from the specific target bacteria. With respect to labeling of the antibodies, these are labeled either directly or indirectly with labels used in other known immunoassays. Direct labels may include fluorescent, chemiluminescent, bioluminescent, radioactive, metallic, biotin or enzymatic molecules. Methods of combining these labels to antibodies or other macromolecules are well known to those in the art. Examples include the methods of Hijmans et al., 1969, An immunofluorescence procedure for the detection of intracellular immunoglobulins, Clin Exp Immunol 4:457-472, for fluorescein isothiocyanate, the method of Goding, 1976, Conjugation of antibodies with fluorochromes: modifications to the standard methods, J Immunol Meth 13:215-226, for tetramethylrhodamine isothiocyanate, and the method of Engvall, 1980, Enzyme immunoassay ELISA and EMIT, J Clin Micro 70:419-439, for enzymes.

These detector antibodies 221 may also be labeled indirectly. In this case the actual detection molecule is attached to a secondary antibody or other molecule with binding affinity for the anti-bacteria cell surface antibody. If a secondary antibody is used it is preferably a general antibody to a class of antibody (IgG and IgM) from the animal species used to raise the anti-bacteria cell surface antibodies. For example, the second antibody may be conjugated to an enzyme, either alkaline phosphatase or to peroxidase. To detect the label, after the bacteria of interest is contacted with the second antibody and washed, the isolated component of the sample is immersed in a solution containing a chromogenic substrate for either alkaline phosphatase or peroxidase.

A chromogenic substrate is a compound that can be cleaved by an enzyme to result in the production of some type of detectable signal which only appears when the substrate is cleaved from the base molecule. The chromogenic substrate is colorless, until it reacts with the enzyme, at which time an intensely colored product is made. Thus, material from the target (e.g., bacterial cells) will become an intense blue/purple/black color, or brown/red. Examples of detection molecules include fluorescent substances, such as 4-methylumbelliferyl phosphate, and chromogenic substances, such as 4-nitrophenylphosphate, 3,3′,5,5′-tetramethylbenzidine and 2,2′-azino-di-[3-ethelbenz-thiazoliane sulfonate]. In addition to alkaline phosphatase and peroxidase, other useful enzymes include β-galactosidase, β-glucuronidase, α-glucosidase, β-glucosidase, α-mannosidase, galactose oxidase, glucose oxidase and hexokinase.

Detection of bacteria of interest using NMR may be accomplished as follows. In the use of NMR as a detection methodology, in which a sample is delivered to a detector coil centered in a magnet, the target of interest, such as a magnetically labeled bacterium, may be delivered by a fluid medium, such as a fluid substantially composed of water. In such a case, the magnetically labeled target may go from a region of very low magnetic field to a region of high magnetic field, for example, a field produced by an about 1 to about 2 Tesla magnet. In this manner, the sample may traverse a magnetic gradient, on the way into the magnet and on the way out of the magnet. As may be seen via equations 1 and 2 below, the target may experience a force pulling into the magnet in the direction of sample flow on the way into the magnet, and a force into the magnet in the opposite direction of flow on the way out of the magnet. The target may experience a retaining force trapping the target in the magnet if flow is not sufficient to overcome the gradient force.

m dot(del B)=F Equation 1

vₜ=−F/(6*p*n*r) Equation 2

where n is the viscosity, r is the bead diameter, F is the vector force, B is the vector field, and m is the vector moment of the bead.

A magnet 231 may be provided that exhibits any desired magnetic field B, such as a non-uniform field. As such, there may be a transverse force that pulls targets to the side of a container or a conduit that provides the sample flow into the magnet. Generally, the time it takes a target to reach the wall of a conduit is associated with the terminal velocity and is lower with increasing viscosity. The terminal velocity is associated with the drag force, which may be indicative of creep flow in certain cases. In general, it may be advantageous to have a high viscosity to provide a higher drag force such that a target will tend to be carried with the fluid flow through the magnet without being trapped in the magnet or against the conduit walls.

Newtonian fluids exhibit characteristic flow patterns. For example, a Newtonian fluid has a parabolic velocity profile across a round pipe, with zero velocity at a wall, and maximal at the center, and having a parabolic characteristic with radius. The velocity decreases in a direction toward the walls, and it is easier to magnetically trap targets near the walls, either with transverse gradients force on the target toward the conduit wall, or in longitudinal gradients sufficient to prevent target flow in the pipe at any position. In order to provide favorable fluid drag force to keep the samples from being trapped in the conduit, it may be advantageous to have a plug flow condition, wherein the fluid velocity is substantially uniform as a function of radial position in the conduit.

When NMR detection is employed in connection with a flowing sample, the detection may be based on a perturbation of the NMR water signal caused by a magnetically labeled target. Detection of beads and target is discussed in Sillerud et al., 2006, 1H NMR detection of superparamagnetic nanoparticles at 1 T using a microcoil and novel tuning circuit, J Mag Res, 181:181-190. In such a case, the sample may be excited at time 0, and after some delay, such as about 50 ms or about 100 ms, an acceptable measurement (based on a detected NMR signal) may be produced. Alternatively, such a measurement may be produced immediately after excitation, with the detection continuing for some duration, such as about 50 ms or about 100 ms. It may be advantageous to detect the NMR signal for substantially longer time durations after the excitation.

By way of example, the detection of the NMR signal may continue for a period of about 2 seconds in order to record spectral information at high-resolution. In the case of parabolic or Newtonian flow, the perturbation excited at time 0 is typically smeared because the water around the perturbation source travels at different velocity, depending on radial position in the conduit. In addition, spectral information may be lost due to the smearing or mixing effects of the differential motion of the sample fluid during signal detection. When carrying out an NMR detection application involving a flowing fluid sample, it may be advantageous to provide plug-like sample flow to facilitate desirable NMR contrast and/or desirable NMR signal detection.

Differential motion within a flowing Newtonian fluid may have deleterious effects in certain situations, such as a situation in which spatially localized NMR detection is desired, as in magnetic resonance imaging. In one example, a magnetic object, such as a magnetically labeled bacterium, is flowed through the NMR detector and its presence and location are detected using MRI techniques. The detection may be possible due to the magnetic field of the magnetic object, since this field perturbs the magnetic field of the fluid in the vicinity of the magnetic object. The detection of the magnetic object is improved if the fluid near the object remains near the object. Under these conditions, the magnetic perturbation may be allowed to act longer on any given volume element of the fluid, and the volume elements of the fluid so affected will remain in close spatial proximity. Such a stronger, more localized magnetic perturbation will be more readily detected using NMR or MRI techniques.

If a Newtonian fluid is used to carry the magnetic objects through the detector, the velocity of the fluid volume elements will depend on radial position in the fluid conduit. In such a case, the fluid near a magnetic object will not remain near the magnetic object as the object flows through the detector. The effect of the magnetic perturbation of the object on the surrounding fluid may be smeared out in space, and the strength of the perturbation on any one fluid volume element may be reduced because that element does not stay within range of the perturbation. The weaker, less-well-localized perturbation in the sample fluid may be undetectable using NMR or MRI techniques.

Certain liquids, or mixtures of liquids, exhibit non-parabolic flow profiles in circular conduits. Such fluids may exhibit non-Newtonian flow profiles in other conduit shapes. The use of such a fluid may prove advantageous as the detection fluid in an application employing an NMR-based detection device. Any such advantageous effect may be attributable to high viscosity of the fluid, a plug-like flow profile associated with the fluid, and/or other characteristic(s) attributed to the fluid that facilitate detection. As an example, a shear-thinning fluid of high viscosity may exhibit a flow velocity profile that is substantially uniform across the central regions of the conduit cross-section. The velocity profile of such a fluid may transition to a zero or very low value near or at the walls of the conduit, and this transition region may be confined to a very thin layer near the wall.

Not all fluids, or all fluid mixtures, are compatible with the NMR detection methodology. In one example, a mixture of glycerol and water can provide high viscosity, but the NMR measurement is degraded because separate NMR signals are detected from the water and glycerol molecules making up the mixture. This can undermine the sensitivity of the NMR detector. In another example, the non-water component of the fluid mixture can be chosen to have no NMR signal, which may be achieved by using a perdeuterated fluid component, for example, or using a perfluorinated fluid component. This approach may suffer from the loss of signal intensity since a portion of the fluid in the detection coil does not produce a signal.

Another approach may be to use a secondary fluid component that constitutes only a small fraction of the total fluid mixture. Such a low-concentration secondary fluid component can produce an NMR signal that is of negligible intensity when compared to the signal from the main component of the fluid, which may be water. It may be advantageous to use a low-concentration secondary fluid component that does not produce an NMR signal in the detector. For example, a perfluorinated or perdeuterated secondary fluid component may be used. The fluid mixture used in the NMR detector may include one, two, or more than two secondary components in addition to the main fluid component. The fluid components employed may act in concert to produce the desired fluid flow characteristics, such as high-viscosity and/or plug flow. The fluid components may be useful for providing fluid characteristics that are advantageous for the performance of the NMR detector, for example by providing NMR relaxation times that allow faster operation or higher signal intensities.

A non-Newtonian fluid may provide additional advantages for the detection of objects by NMR or MRI techniques. As one example, the objects being detected may all have substantially the same velocity as they go through the detection coil. This characteristic velocity may allow simpler or more robust algorithms for the analysis of the detection data. As another example, the objects being detected may have fixed, known, and uniform velocity. This may prove advantageous in devices where the position of the detected object at later times is needed, such as in a device that has a sequestration chamber or secondary detection chamber down-stream from the NMR or MRI detection coil, for example.

In an exemplary embodiment, sample delivery into and out of a 1.7 T cylindrical magnet using a fluid delivery medium containing 0.1% to 0.5% Xanthan gum in water was successfully achieved. Such delivery is suitable to provide substantially plug-like flow, high viscosity, such as from about 10 cP to about 3000 cP, and good NMR contrast in relation to water. Xanthan gum acts as a non-Newtonian fluid, having characteristics of a non-Newtonian fluid that are well known in the art, and does not compromise NMR signal characteristics desirable for good detection in a desirable mode of operation.

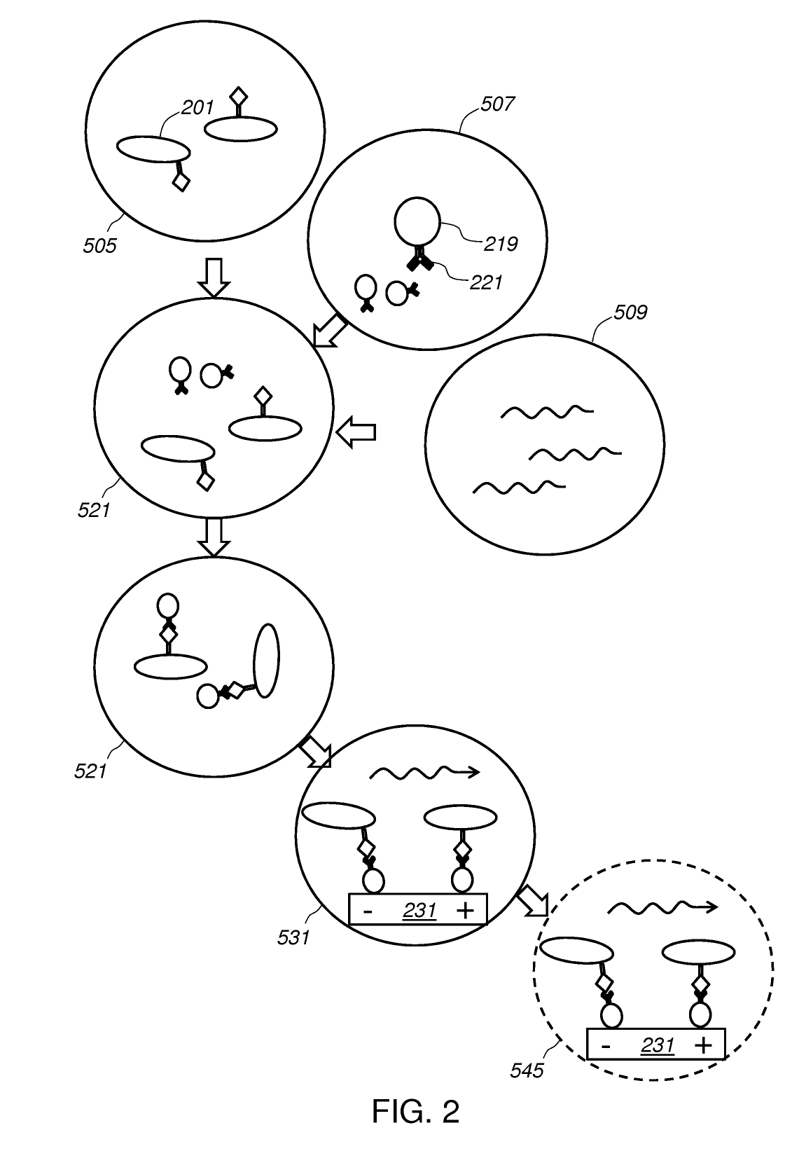

FIG. 2 illustrates methods of detecting a target analyte 201 using a fluidic device according to certain embodiments. A sample is delivered, for example from a collection tube 505, into a chamber 521. A binding buffer is delivered from reservoir 509 to chamber 521 and particles 219 with binding moiety 221 are delivered (for example, from ampoule 507) to chamber 521. Reservoir 509 becomes pressurized by pneumatic passages, or tubes, in pneumatic interface 511. The binding buffer may be formulated to enhance binding of the binding moiety to the target analyte, reduce formation of particle aggregates, or both. The sample is incubated in chamber 521 until binding moiety 221 binds target analyte 221. Note that in certain embodiments, the bead buffer mixture is optionally flowed through tube 505 and binding also occurs there.

Target analyte 201 is then isolated from a remainder of the sample. In some embodiments, the bead-bound target analyte 201 is transferred into magnetic trap 531, and is isolated using magnet 231 (e.g., using magnetic isolation techniques as described above). Moreover, the binding buffer, a wash buffer, or both, can be used—serially or in combination—to wash away components of the sample. In certain embodiments, bead-bound target analyte 201 is transferred to magnetic concentrator 545 where the target is further concentrated (e.g., washed by a wash buffer and concentrated to a desired concentration). In some embodiments, concentrating target analyte 201 involves flushing concentrator chamber 545 with the wash buffer. In certain alternative embodiments, a single chamber is used, and a wash solution is flushed through the chamber after the incubation with the buffer solution. Target analyte 201 can then be analyzed, for example, by replacing the wash buffer with a cell maintenance solution to keep cells alive or with a lysis buffer to extract a component of the analyte.

In certain embodiments, methods of the invention are useful for direct detection of bacteria from blood. Such a process is described here. Sample is collected in sodium heparin tube by venipuncture, acceptable sample volume is about 1 mL to 10 mL. Sample is diluted with binding buffer and superparamagnetic particles having target-specific binding moieties are added to the sample, followed by incubation on a shaking incubator at 37° C. for about 30 min to 120 min. Alternative mixing methods can also be used. In a particular embodiment, sample is pumped through a static mixer, such that reaction buffer and magnetic beads are added to the sample as the sample is pumped through the mixer. This process allows for efficient integration of all components into a single fluidic part, avoids moving parts and separate incubation vessels and reduces incubation time.

Capture of the labeled targets allows for the removal of blood components and reduction of sample volume from 30 mL to 5 mL. The capture is performed in a variety of magnet/flow configurations. In certain embodiments, methods include capture in a sample tube on a shaking platform or capture in a flow-through device at flow rate of 5 mL/min, resulting in total capture time of 6 min.

After capture, the sample is washed with wash buffer including heparin to remove blood components and free beads. The composition of the wash buffer is optimized to reduce aggregation of free beads, while maintaining the integrity of the bead/target complexes.

In some embodiments, the detection method is based on a miniature NMR detector tuned to the magnetic resonance of water. When the sample is magnetically homogenous (no bound targets), the NMR signal from water is clearly detectable and strong. The presence of magnetic material in the detector coil disturbs the magnetic field, resulting in reduction in water signal. One of the primary benefits of this detection method is that there is no magnetic background in biological samples which significantly reduces the requirements for stringency of sample processing. In addition, since the detected signal is generated by water, there is a built-in signal amplification which allows for the detection of a single labeled bacterium. This method provides for isolation and detection of as low as or even lower than 1 CFU/ml of bacteria in a blood sample.

In certain aspects, the invention provides fluidic devices and systems for the analysis of target analytes. Any suitable device or system may be used. In general, the devices comprise an input channel, an output channel, a chamber, a magnetic assembly, and a lysing device. The input channel and output channel are in fluid communication with the chamber and the magnetic assembly is adapted to capture a magnetic particle inputted into the input channel onto a surface of the chamber. The lysing device is adapted to lyse a target bound to the magnetic particle. In one illustrative embodiment, the device is a fluidic cartridge.

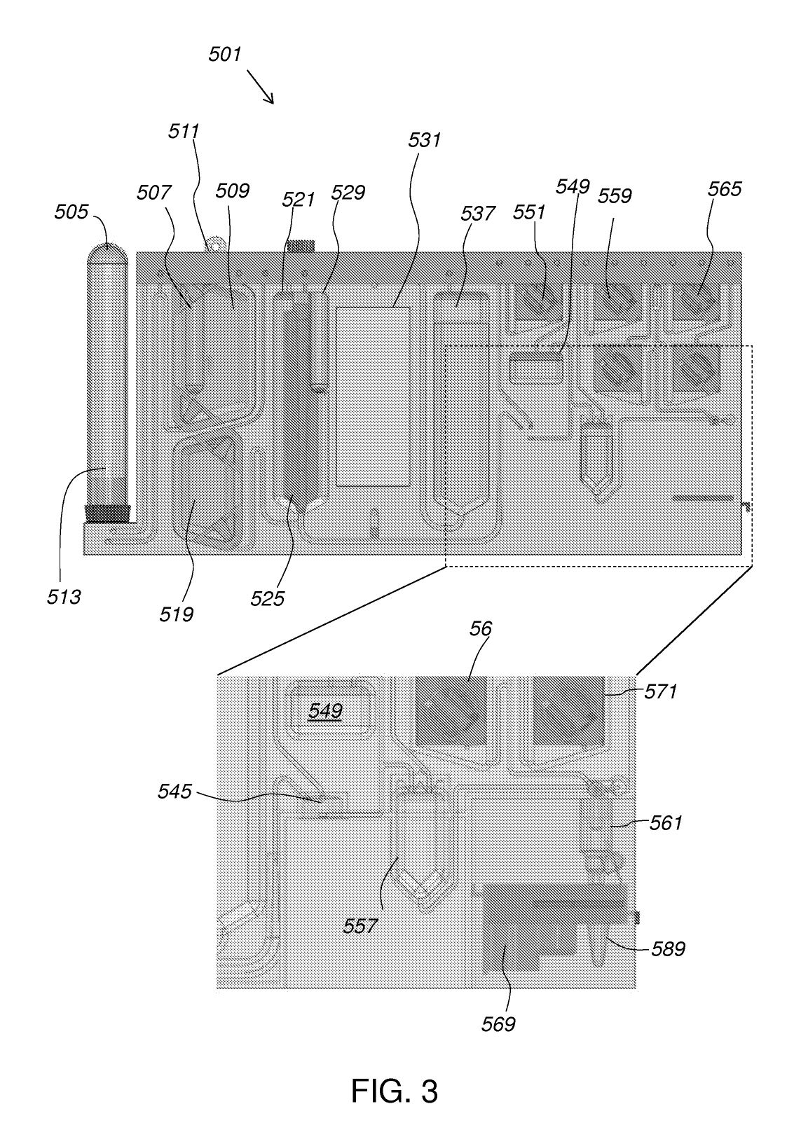

FIG. 3 illustrates a fluidic cartridge 501 according to certain embodiments. Fluidic cartridge 501 is provided to operate with sample collection tube 505 (e.g., a vacutainer). Cartridge 501 includes long needle 513, extending from a receiving member of cartridge 501. Needle 513 defines a hollow member to penetrate into an interior of tube 505 when tube 505 is inserted thereon. Magnetic beads 219 may be stored in magnetic bead ampule 507 shown disposed within bead buffer 509. Pneumatic interface 511 provides pressure to flow sample, solutions, and buffers through channels of cartridge 501 and may optionally be used to rupture ampoules such as magnetic bead ampoule 507 (and plant pathogen ampoule 529). When operation is begun and tube 505 containing a sample is inserted, magnetic bead ampoule 507 is crushed, introducing beads 219 to bead buffer 509. Beads 219 and buffer 509 may be mixed using agitation provided by air from pneumatic interface 511, to produce a bead mixture.

Pressure from pneumatic interface 511 forces sample from tube 505 into mixing chamber 521. The bead mixture is forced through long needle 513 to rinse the inside of tube 505. The bead mixture is then sent to mixing chamber 521 to mix with the sample. This step may be repeated, sending more bead mixture from bead buffer 509, through tube 505, to mixing chamber 521, until tube 505 is evacuated of target and a desired proportion (e.g., 2:1) of bead mixture to sample is present in mixing chamber 521.

Mixing paddle 525 is used to mix the contents of chamber 521, agitating the sample and beads. Air pressure from pneumatic interface 511 then forces the mixture into magnetic trap 531. Optionally, the mixture can be pushed back, from magnetic trap 531 to mixing chamber 521, and the cycle repeated any number times until mixing chamber 521 is satisfactorily evacuated of target pathogens 201 and the bead-bound target pathogens 201 as well as the other components of the sample are in magnetic trap 431.

Then, magnetic trap 431 is evacuated of the other components of the sample, leaving magnetic particles 219 bound to magnets therein. The waste fluid is pushed back through mixing chamber 521 and discarded. Wash buffer 519 is then forced into magnetic trap 531, filling it. The wash buffer can then be pushed back to mixing chamber 521 to ensure good washing of beads 219. Wash buffer is sent back into magnetic trap 531 and held there.

Magnets can then be moved away from magnetic trap 531, allowing beads 219 to re-suspend in wash buffer 519 within trap 531. At this point, target pathogens 201 have been extracted from the original sample and held in wash buffer 519. Wash buffer 519 can then be pushed through magnetic concentrator 545 to waste chamber 549.

Turning now to the inset portion of FIG. 3, magnetic concentrator 545 can be seen to be in fluid communication with magnetic trap 531. As wash buffer 519 flows through concentrator 545, beads 219 are captured in concentrator 545 while the remainder of buffer 519 is passed on to waste chamber 549. Original target analytes 201 are thus concentrated in concentrator 545.

The contents of concentrator 545 may be processed according to a desired output. Where target analyte 201 includes cells (e.g., a microbial pathogen) whether live cells or an extracted cellular component is intended can be used to determine a following step. If live cells are intended, the magnet is removed from magnetic concentrator 545 and buffer 551 (here, a live cell buffer) is introduced through concentrator 545 and used to flush the cells into output vial 589.

If, for example, extracted DNA is intended, buffer 551 is a lysis buffer and is introduced into magnetic concentrator 545. The magnet is removed from concentrator 545 and a sonicator may be applied to a wall of the chamber of concentrator 545. Optionally, beads may be included for bead-bashing to aid in lysis. The sonicator or other lysis means is activated and the target cells 201 are lysed. In certain embodiments, a probe of a sonicator extends into the chamber, where it delivers vibrations into the liquid medium surrounding the captured complexes. In other embodiments, the sonication transducer is brought in contact with the chamber by way of a structural interface. For example, the structural interface may constitute the floor or the ceiling of the chamber. The sonication transducer vibrates the structural interface such that lysis of the targets captured in the chamber is achieved.

Lysate is then pushed into pre-column mixer 557. DNA binding buffer 559 is added to pre-column mixer 557 (optionally agitated by bubbling air). The contents of pre-column mixer 557 is then forced through the DNA extraction column 561 and the elutant is discarded as waste. DNA extraction may be completed using washes from first column wash 565, second column wash 569, and water 571. Air from pneumatic interface 511 can be forced through DNA extraction column 561 to remove volatile organic compounds. Water 571 can be used to rinse the purified DNA (optionally including the use of a de-binding buffer or a modulator of stringency) into output vial 589.

Coordination of the on-cartridge steps can be supported by an operations device, such as a bench-top electro-mechanical device. The timing of pneumatic injections, the breaking of ampoules, and the piercing of reagent reservoirs can be coordinated manually, or by a computer program or mechanical system. Cartridge 501 can include any suitable materials, shape, or dimensions. For example, in some embodiments, fluids are handled in macrofluidic (e.g., fluidic) environments up until entered into magnetic concentrator 545 and are handled according to microfluidic principles thereafter. In general, microfluidic may refer to sub-microliter volumes. Macrofluidic, or fluidic, can refer to fluidic volumes that are not microfluidic.

Generally, microfluidics relates to small sample volumes and small channel pathways. For example, microfluidic volumes are normally below 1 mL, or on the microliter (μL) scale or smaller, for example, nL (nanoliters) and pL (picoliters). As used herein, microfluidic volumes relate to volumes less than 1 mL. In addition, microfluidics relates to small channel pathways on the micrometer scale. As used herein, microfluidic channels within systems of the invention refer to channels that have channel heights and/or widths equal to or less than 500 μm. See “Microfluidics and Nanofluidics: Theory and Selected Applications,” Kleinstruer, C., John Wiley & Sons, 2013, which is incorporated by reference. The channel height or width is defined as the height or width of the path that the sample volume must pass through within the cartridge. Comparatively, macrofluidics volumes relate to volumes greater than the microliter (μL) scale, for example, sample volumes on the milliliter (mL) scale. As used herein, macrofluidic volumes are volumes of 1 mL or greater. Macrofluidic channels within systems of the invention are channels having channel heights and/or widths of greater than 500 μm.

Other macrofluidic components are chambers, reservoirs, traps, mixers, etc. Such macrofluidic components are dimensioned to hold 1 mL or more of fluid. For example, the individual volume can range without limitation from about 10 to about 50 mL. Other microfluidic components are chambers, reservoirs, traps, mixers, etc. Such microfluidic components are dimensioned to hold less than 1 mL of fluid. For example, the individual volumes can range without limitation from about 1 μL to about 500 μL.

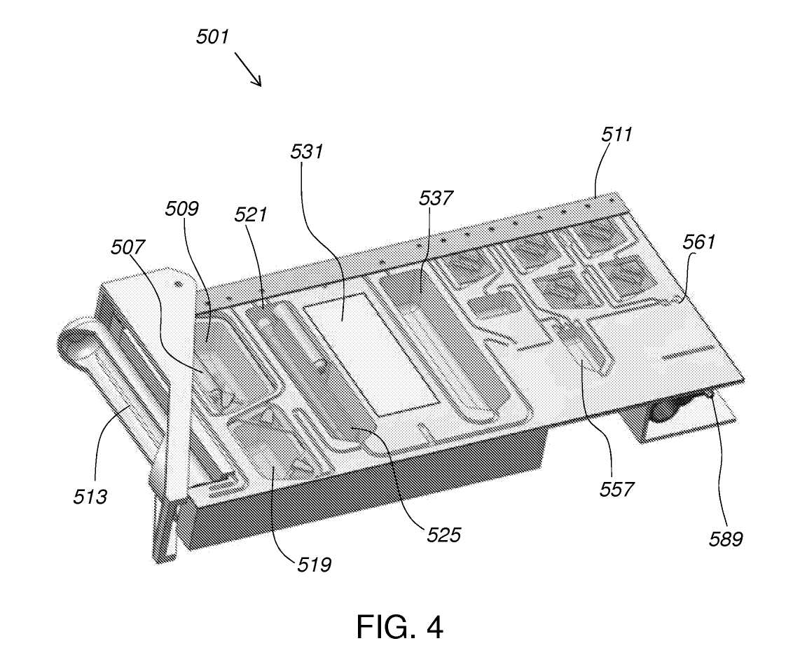

FIG. 4 gives a perspective view of an exemplary cartridge 501 according to certain embodiments. Methods for manufacturing and operating fluidic systems are known and discussed in Fredrickson and Zan, 2004, Macro-to-micro interfaces for microfluidic devices, Lab Chip 4(6):526-33; U.S. Pat. No. 8,105,783; U.S. Pat. No. 7,785,869; U.S. Pat. No. 7,745,207; U.S. Pat. No. 7,553,647; U.S. Pub. 2009/0227005; U.S. Pub. 2008/0241000; and U.S. Pub. 2008/0003564, the contents of each of which are incorporated by reference in their entirety for all purposes.

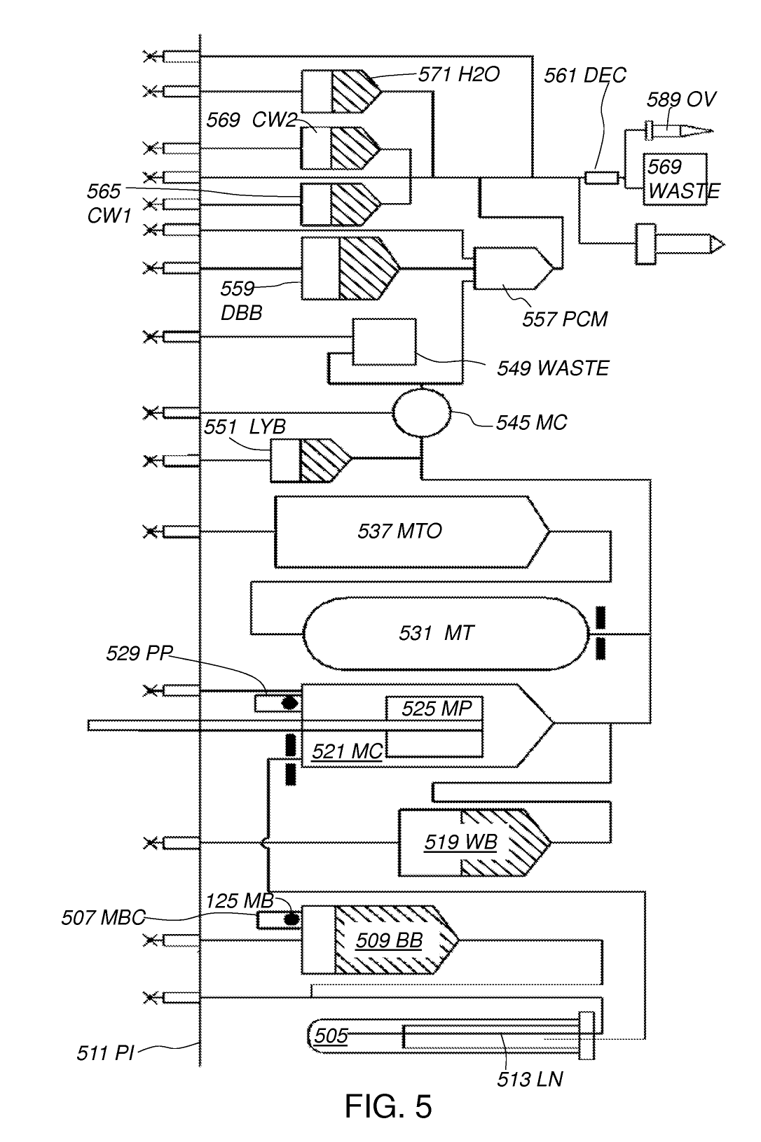

FIG. 5 gives a schematic diagram of elements and steps of the invention as discussed above with respect to FIG. 3.

While discussed above as magnetic beads, substrate 219 for capture of pathogens 201 may be the floor of a chamber such as, for example, trap chamber 531. In other embodiments, the capture surface is the ceiling. The captured pathogens 201 can there be washed to remove non-specific analytes and unbound entities.

In certain embodiments, fluidic cartridge 501 is designed to be disposable. In this instance, once pathogens 201 have been lysed and the eluate collected, cartridge 501 can be discarded. In this manner, each cartridge is intended for a single use, and the potential for cross-contaminating pathogens due to repeated use is eliminated.

The cartridge can be prepared from any material in the art suitable for containing liquids and withstanding the rigors of sonication. In certain aspects, the entire cartridge is made of plastic or an acrylic plastic polymer. In other aspects, the bottom or top cover of the cartridge can be prepared from a film such as the biaxially-oriented polyethylene terephthalate (BoPET) sold under the trademark MYLAR while the rest of the cartridge is made of plastic or an acrylic plastic polymer. In certain embodiments, the BoPET film constitutes the aforementioned structural interface in contact with the sonicator transducer.

In certain embodiments, the cartridge is connected to a fluidic device or system configured to flow liquids into and out of the cartridge. The fluidic system can comprise a pump that delivers liquid, air, gas, beads, reagents, electricity or other signals, light, power or a combination thereof to the cartridge. The fluidic system can have a first tubing line connected to the inlet of the input channel, for flowing fluid (e.g., gas or liquid) into the cartridge. The other end of the first tubing line is connected to the pump. A second tubing line for fluid leaving the cartridge is connected to the outlet of the output channel. The other end of the second tubing line may also be connected to the pump or to a container for collecting exiting fluid. The fluidic system facilitates the delivery of the target/magnetic particle complexes into the chamber, as well as any washing of captured complexes. The collection of lysate is also facilitated by the fluidic system. In some embodiments, all steps are performed on a single cartridge 501. For example, sample collection tube 505 may be loaded into cartridge 501 and all of the described steps may be performed “on-chip” including delivering cells or an extracted material into an output vial. In certain embodiments, all of the on-chip steps are substantially automated.

Devices of the invention may also include a detection module. The detection module is a component in which molecules, cells, or other particles are to be detected, identified, measured or interrogated on the basis of at least one predetermined characteristic. The molecules, cells, or other particles can be examined one at a time, and the characteristic is detected or measured optically, for example, by testing for the presence or amount of a label. In some aspects, the detection module is in connection with one or more detection apparatuses. The detection apparatuses can be optical or electrical detectors or combinations thereof. Examples of suitable detection apparatuses include optical waveguides, microscopes, diodes, light stimulating devices (e.g., lasers), photomultiplier tubes, optically transmissive cuvettes, fluidic chambers with electrodes to sense impedance changes, and processors (e.g., computers and software), and combinations thereof, which cooperate to detect a signal representative of a characteristic, marker, probe, label, or reporter, and to determine and direct the measurement or sorting action at the sorting module. However, other detection techniques can be used as well. In some embodiments, the target analyte is detected by a change in electrical conductivity or impendence in a fluidic medium. Measuring impedance or resistance for analyte detection is discussed in U.S. Pat. No. 6,990,849; U.S. Pub. 2011/0136102; U.S. Pub. 2011/0020459; and U.S. Pub. 2007/0238112, the contents of each of which are incorporated by reference.

Devices of the invention may include a computer component. For example, a user interface may be provided with input/output mechanisms (monitor, keyboard, touchscreen, etc.) coupled to a memory and a processor (e.g., a silicone chip and a solid-state or magnetic hard drive). Input/output mechanisms may be included for data, such as a USB port, Ethernet port, or Wi-Fi card or a hardware connection to a detection module, fluidic chip and pneumatic interface, or both.

In certain aspects, the detection module is in fluid connection with the fluidic cartridge. For example, the outlet of the fluidic cartridge may be connected to the detection module by means of a tubing line. In this manner, lysate leaving the cartridge can enter the detection module wherein the contents of the lysate can be detected and analyzed.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

### Example 1

### Sample

Blood samples from healthy volunteers were spiked with clinically relevant concentrations of bacteria (1-10 CFU/mL) including both laboratory strains and clinical isolates of the bacterial species most frequently found in bloodstream infections.

### Example 2

### Antibody Preparation

In order to generate polyclonal, pan-Gram-positive bacteria-specific IgG, a goat was immunized by first administering bacterial antigens suspended in complete Freund's adjuvant intra lymph node, followed by subcutaneous injection of bacterial antigens in incomplete Freund's adjuvant in 2 week intervals. The antigens were prepared for antibody production by growing bacteria to exponential phase (OD₆₀₀=0.4-0.8). Following harvest of the bacteria by centrifugation, the bacteria was inactivated using formalin fixation in 4% formaldehyde for 4 hr at 37° C. After 3 washes of bacteria with PBS (15 min wash, centrifugation for 20 min at 4000 rpm) the antigen concentration was measured using bicinchoninic (BCA) assay and the antigen was used at 1 mg/mL for immunization. In order to generate Gram-positive bacteria-specific IgG, several bacterial species were used for inoculation: Staphylococcus aureus, Staphylococcus epidermidis, Enterococcus faecium and Enterococcus faecalis.

The immune serum was purified using affinity chromatography on a protein G sepharose column (GE Healthcare), and reactivity was determined using ELISA. Antibodies cross-reacting with Gram-negative bacteria and fungi were removed by absorption of purified IgG with formalin-fixed Gram-negative bacteria and fungi. The formalin-fixed organisms were prepared similar to as described above and mixed with IgG. After incubation for 2 hours at room temperature, the preparation was centrifuged to remove bacteria. Final antibody preparation was clarified by centrifugation and used for the preparation of antigen-specific magnetic beads.

### Example 3

### Preparation of Antigen-Specific Magnetic Beads

Superparamagnetic beads were synthesized by encapsulating iron oxide nanoparticles (5-15 nm diameter) in a latex core and labeling with goat IgG. Ferrofluid containing nanoparticles in organic solvent was precipitated with ethanol, nanoparticles were resuspended in aqueous solution of styrene and surfactant Hitenol BC-10, and emulsified using sonication. The mixture was allowed to equilibrate overnight with stirring and filtered through 1.2 and 0.45 μm filters to achieve uniform micelle size. Styrene, acrylic acid and divynilbenzene were added in carbonate buffer at pH 9.6. The polymerization was initiated in a mixture at 70° C. with the addition of K₂S₂O₈ and the reaction was allowed to complete overnight. The synthesized particles were washed 3 times with 0.1% SDS using magnetic capture, filtered through 1.2, 0.8, and 0.45 μm filters and used for antibody conjugation.

The production of beads resulted in a distribution of sizes that may be characterized by an average size and a standard deviation. In the case of labeling and extracting of bacteria from blood, the average size for optimal performance was found to be between 100 and 350 nm, for example between 200 nm to 250 nm.

The purified IgG were conjugated to prepared beads using standard chemistry. After conjugation, the beads were resuspended in 0.1% BSA which is used to block non-specific binding sites on the bead and to increase the stability of bead preparation.

### Example 4

### Labeling of Rare Cells Using Excess of Magnetic Nanoparticles

Bacteria, present in blood during blood-stream infection, were magnetically labeled using the superparamagnetic beads prepared in Example 3 above. The spiked samples as described in Example 1 were diluted 3-fold with a Tris-based binding buffer and target-specific beads, followed by incubation on a shaking platform at 37° C. for up to 2 hr. After incubation, the labeled targets were magnetically separated followed by a wash step designed to remove blood products. See example 5 below.

### Example 5

### Magnetic Capture of Bound Bacteria

Blood including the magnetically labeled target bacteria and excess free beads were injected into a flow-through capture cell with a number of strong rare earth bar magnets placed perpendicular to the flow of the sample. With using a flow chamber with flow path cross-section 0.5 mm×20 mm (h×w) and 7 bar NdFeB magnets, a flow rate as high as 5 mL/min was achieved. After flowing the mixture through the channel in the presence of the magnet, a wash solution including heparin was flowed through the channel. The bound targets were washed with heparin-containing buffer one time to remove blood components and to reduce formation of magnetic particle aggregates. In order to effectively wash bound targets, the magnet was removed and captured magnetic material was resuspended in wash buffer, followed by re-application of the magnetic field and capture of the magnetic material in the same flow-through capture cell.

Removal of the captured labeled targets was possible after moving magnets away from the capture chamber and eluting with flow of buffer solution.

What is claimed is:

1. A device for capturing a target in a sample, the device comprising:
a receiving member configured to receive a vessel;
a fluidic network, wherein the fluidic network is configured for isolation of a target from a sample; and
a hollow member configured to enter the vessel and to displace the sample from the vessel and into the fluidic network that is coupled to the hollow member, wherein the hollow member is connected to a pressurizable fluid reservoir.

2. The device according to claim 1, wherein the fluidic network comprises a target capture chamber.

3. The device according to claim 2, wherein the target capture chamber comprises magnets configured to generate a magnetic field within the chamber.

4. The device according to claim 3, wherein the fluidic network further comprises a mixing chamber located prior to the capture chamber and a concentrating chamber located after the capture chamber.

5. The device according to claim 4, wherein the fluidic network comprises a plurality of reservoirs after the capture chamber.

6. The device according to claim 5, wherein macrofluidic channels of the fluidic network connect the hollow member, the capture chamber, the mixing chamber, and the concentrating chamber.

7. The device according to claim 6, wherein microfluidic channels connect the plurality of reservoirs and the concentrating chamber.

8. The device according to claim 1, further comprising one or more air channels connected to the pressurizable fluid reservoir.

9. The device according to claim 1, wherein the vessel is a vacutainer.

10. The device according to claim 1, further comprising an outlet after the concentrating chamber.

11. The device according to claim 1, wherein the pressurizable fluid reservoir comprises an breakable ampule.

12. The device according to claim 11, wherein the ampule comprises a moiety that specifically binds the target.

13. The device according to claim 12, wherein the moiety is bound to a magnetic bead.

14. A method for capturing a target from a sample, the method comprising:
transferring a sample comprising a target from a vessel to a fluidic network;
flushing the vessel with a solution comprising a moiety that specifically binds the target, which solution is also transferred to the fluidic network;
mixing the sample and the solution in the fluidic network so that the moiety binds the target, thereby forming target/moiety complexes; and
isolating the target/moiety complexes from the sample in the fluidic network.

15. The method according to claim 14, wherein the moiety is bound to magnetic particles.

16. The method according to claim 15, wherein mixing comprises, flowing the sample into a mixing chamber configured to mix the sample and the solution.

17. The method according to claim 16, wherein isolating comprises flowing the sample and solution from the mixing chamber into a target capture chamber that comprises a plurality of magnetics that are configured to generate a magnetic field within the chamber.

18. The method according to claim 14, further comprising:
exposing the isolated the target/moiety complexes to a buffer solution; and
collecting the isolated target/moiety complexes.

19. The method according to claim 14, wherein the target is a microorganism.

20. The method according to claim 19, further comprising:
exposing the isolated the microorganism/moiety complexes to a lysis solution, thereby lysing the microorganisms; and
collecting nucleic acid released from the lysed microorganisms.
